(19) United States
(12) Patent Application Publication
(10) Pub. No.: US 2022/0386981 A1
(43) Pub. Date: Dec. 8, 2022

(54) INFORMATION PROCESSING SYSTEM AND INFORMATION PROCESSING METHOD

(71) Applicant: CANON KABUSHIKI KAISHA, Tokyo (JP)

(72) Inventor: Shohei Hosoda, Tokyo (JP)

(21) Appl. No.: 17/748,195

(22) Filed: May 19, 2022

(30) Foreign Application Priority Data
Jun. 4, 2021 (JP) 2021-094576

(51) Int. Cl. A61B 6/00; G06T 7/00; G06T 7/70; G16H 30/20; A61B 6/14

(52) U.S. Cl. CPC A61B 6/5247; G06T 7/0012; G06T 7/70; G16H 30/20; A61B 6/14; A61B 6/463; A61B 6/5294; G06T 2207/30244; G06T 2207/10116; G06T 2207/30036

(57) ABSTRACT

An information processing system comprises: an information processor capable of transmitting and receiving data; and an estimation unit that estimates dental notations of teeth in each of visible light images and X-ray images of oral cavities input through the information processor and estimates image shooting direction of each of the visible light images and the X-ray images. The information processor adds the dental notations and the image sensing direction to each of the visible light images and the X-ray images as metadata, and manages the visible light images and the X-ray images by associating the visible light images and the X-ray images using the metadata.

### BACKGROUND OF THE INVENTION

### Field of the Invention

The present invention relates to an information processing system and an information processing method, and particularly to a technique for associating visible light images with X-ray images in a dental examination.

### Description of the Related Art

In dental examinations, photographs (images) of an oral cavity are taken at various angles with a plurality of different image capturing apparatuses (digital cameras, X-ray image capturing apparatuses, etc.), and the taken images are used to determine the treatment policy and to observe the progress. Japanese Patent Laid-Open No. 2018-84982 discloses a technique of extracting a feature amount from a plurality of images and generating a high-quality image by synthesizing the images.

The prior art disclosed in Japanese Patent Laid-Open No. 2018-84982 assumes that images are associated one-to-one.

However, in dental examinations, five visible light images are generally taken by a digital camera from five directions of “upper, lower, left, right, front”, which is called a 5-sheet method, and ten X-ray images are usually taken by the X-ray image capturing apparatus from 10 directions, which is called a 10-sheet method. As described above, there are many cases where the numbers of images differ between the visible light images and the X-ray images, and it is difficult to associate between those images by the conventional technique disclosed in Japanese Patent Laid-Open No. 2018-84982.

### SUMMARY OF THE INVENTION

The present invention has been made in consideration of the above situation, and enables management by associating between visible light images and X-ray images taken in different ways.

According to the present invention, provided is an information processing system comprising one or more processors and/or circuitry which functions as: an information processor capable of transmitting and receiving data; and an estimation unit that estimates dental notations of teeth in each of visible light images and X-ray images of oral cavities input through the information processor and estimates image shooting direction of each of the visible light images and the X-ray images, wherein the information processor adds the dental notations and the image sensing direction to each of the visible light images and the X-ray images as metadata, and manages the visible light images and the X-ray images by associating the visible light images and the X-ray images using the metadata.

Further, according to the present invention, provided is an information processing method comprising: inputting visible light images and X-ray images of oral cavities; and estimating dental notations of teeth in each of the visible light images and the X-ray images and estimating image shooting direction of each of the visible light images and the X-ray images, the dental notations and the image sensing direction are added to each of the visible light images and the X-ray images as metadata, and the visible light images and the X-ray images are managed by associating the visible light images and the X-ray images using the metadata.

Furthermore, according to the present invention, provided is a non-transitory computer-readable storage medium, the storage medium storing a program that is executable by the computer, wherein the program includes program code for causing the computer to function as an image processing system comprising: an information processor capable of transmitting and receiving data; and an estimation unit that estimates dental notations of teeth in each of visible light images and X-ray images of oral cavities input through the information processor and estimates image shooting direction of each of the visible light images and the X-ray images, wherein the information processor adds the dental notations and the image sensing direction to each of the visible light images and the X-ray images as metadata, and manages the visible light images and the X-ray images by associating the visible light images and the X-ray images using the metadata.

Further features of the present invention will become apparent from the following description of exemplary embodiments (with reference to the attached drawings).

### BRIEF DESCRIPTION OF THE DRAWINGS

The accompanying drawings, which are incorporated in and constitute a part of the specification, illustrate embodiments of the invention, and together with the description, serve to explain the principles of the invention.

FIG. 1 is a diagram showing a system configuration according to an embodiment of the present invention.

FIG. 2 is a block diagram showing a hardware configuration of each apparatus according to the embodiment.

FIG. 3 is a block diagram showing a functional configuration of each apparatus according to the embodiment.

FIG. 4 is a conceptual diagram of an estimation model according to the embodiment.

FIG. 5 is a diagram showing a data flow in the system according to the embodiment.

FIGS. 6A and 6B illustrate a flowchart showing information processing according to a first embodiment.

FIGS. 7A and 7B are diagrams for explaining how to associate images according to the first embodiment.

FIG. 8 is a diagram showing an example of a user interface according to the first embodiment.

FIGS. 9A and 9B illustrate a flowchart showing information processing according to a second embodiment.

FIGS. 10A to 10D are explanatory diagrams for adding progress information according to the second embodiment.

### DESCRIPTION OF THE EMBODIMENTS

Hereinafter, embodiments will be described in detail with reference to the attached drawings. Note, the following embodiments are not intended to limit the scope of the claimed invention, and limitation is not made an invention that requires a combination of all features described in the embodiments. Two or more of the multiple features described in the embodiments may be combined as appropriate. Furthermore, the same reference numerals are given to the same or similar configurations, and redundant description thereof is omitted.

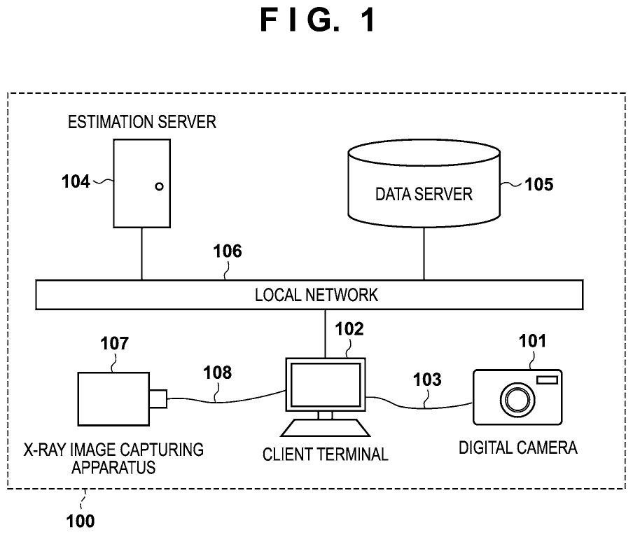

An information processing system 100 to which the present invention can be applied will be described with reference to FIG. 1.

The information processing system 100 includes a digital camera 101 used by a user such as a nurse or a doctor, an X-ray image capturing apparatus 107 used by the user, and a client terminal 102 which is connected to the digital camera 101 and the X-ray image capturing apparatus 107 and is capable of transmitting and receiving data to/from the digital camera 101 and the X-ray image capturing apparatus 107.

Communication between the digital camera 101 and the client terminal 102 is carried out via a first communication path 103 such as USB. Further, communication between the X-ray image capturing apparatus 107 and the client terminal 102 is carried out via a second communication path 108 such as USB. The first communication path 103 and the second communication path 108 may use a wired communication such as USB, or a wireless communication such as Wi-Fi (registered trademark) or Bluetooth (registered trademark).

Furthermore, the information processing system 100 includes an estimation server 104 capable of performing image analysis and estimating the dental notations and the conditions of the teeth in the image (state of dental caries, etc.), and the image sensing direction (from which direction the image was sensed), and a data server 105 for managing images.

A local network 106 connects the client terminal 102, an estimation server 104, and a data server 105 to enable mutual communication.

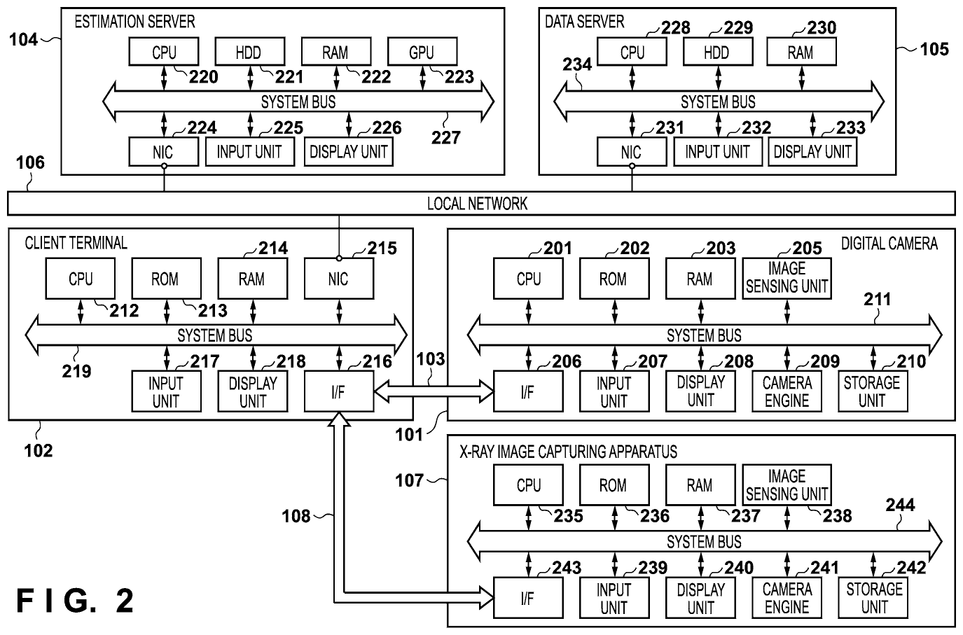

Next, the hardware configuration of each device constituting the system shown in FIG. 1 will be described with reference to FIG. 2.

First, the configuration of the digital camera 101 will be explained.

A CPU 201 controls the entire digital camera 101 and also controls the power supply. A ROM 202 stores programs and data used by the CPU 201 for operating the digital camera 101. A RAM 203 is used to temporarily expand the program read from the ROM 202 by the CPU 201, execute the program expanded thereon, and temporarily hold the operational data.

An image sensing unit 205 is used for sensing images, and includes an image sensor with which the digital camera 101 captures images (visible light images). The CPU 201 detects an image sensing instruction by the user, and in response to the image sensing instruction as a trigger, the image sensing unit 205 executes an image sensing operation. An I/F unit 206 is used for exchanging data between the digital camera 101 and the client terminal 102 via the first communication path 103. An input unit 207 includes a switch for designating an operation mode to the digital camera 101, a motion sensor for detecting motion for performing an image stabilization function, focus control and exposure compensation.

A display unit 208 displays an image/images being sensed or having been captured by the image sensor of the image sensing unit 205, an operating state of the digital camera 101, and so forth.

A camera engine 209 processes an image captured by the image sensor of the image sensing unit 205, and performs image processing for displaying an image stored in a storage unit 210, which will be described later, on the display unit 208.

The storage unit 210 stores image data of still images and moving images captured by the digital camera 101.

A system bus 211 connects the constituents 201 to 210 of the digital camera 101 described above.

Next, the configuration of the X-ray image capturing apparatus 107 will be described.

A CPU 235 controls the entire X-ray image capturing apparatus 107 and also controls the power supply. A ROM 236 stores programs and data used by the CPU 235 for operating the entire X-ray image capturing apparatus 107. A RAM 237 is used to temporarily expand the program read from the ROM 236 by the CPU 235, execute the program expanded thereon, and temporarily hold the operational data.

An image sensing unit 238 is used for sensing images, and includes an image sensor for capturing images (X-ray images). The CPU 235 detects an image sensing instruction by the user, and in response to the image sensing instruction as a trigger, the image sensing unit 238 executes an image sensing operation. An I/F unit 243 is used for exchanging data between the X-ray image capturing apparatus 107 and the client terminal 102 via the second communication path 108. An input unit 239 includes a switch for designating an operation mode to the X-ray image capturing apparatus 107.

A display unit 240 displays an image/images captured by the image sensor of the image sensing unit 238, an operating state of the X-ray image capturing apparatus 107, and so forth.

A camera engine 241 processes an image captured by the image sensor of the image sensing unit 238, and performs image processing for displaying an image stored in a storage unit 242, which will be described later, on the display unit 240.

The storage unit 242 stores the image data of X-ray images captured by the X-ray image capturing apparatus 107.

A system bus 244 connects the constituents 235 to 243 of the X-ray image capturing apparatus 107.

Next, the configuration of the client terminal 102 will be described.

A CPU 212 controls the entire client terminal 102. A HDD 213 stores programs and electronic medical record data used by the CPU 212 for operating the client terminal 102. A RAM 214 is used to temporarily expand the program read from the HDD 213 by the CPU 212, execute the program expanded thereon, and temporarily hold the operational data.

A NIC 215 is used to communicate with the estimation server 104 and the data server 105 via the local network 106. An I/F unit 216 is used to exchange data between the client terminal 102 and the digital camera 101 via the first communication path 103, and the X-ray image capturing apparatus 107 via the second communication path 108. An input unit 217 is composed of a keyboard, a mouse, and the like for operating the client terminal 102.

A display unit 218 displays input statuses and the like of the client terminal 102.

A system bus 219 connects the constituents 212 to 218 of the client terminal 102 described above.

Next, the configuration of the estimation server 104 will be described.

A CPU 220 controls the entire estimation server 104. A HDD 221 stores programs and data used by the CPU 220 for operating the estimation server 104. A RAM 222 is used to temporarily expand the program read from the HDD 221 by the CPU 220, execute the program expanded thereon, and temporarily hold the operational data.

A GPU 223 is specialized in data calculation processing so that calculation for image processing and matrix calculation can be performed at high speed, and a large amount of data can be processed. Since the GPU 223 can perform efficient calculation by processing data in parallel, it is effective to use the GPU 223 when performing estimation using an estimation model. Therefore, in the present embodiment, the GPU 223 is used in addition to the CPU 220 for performing estimation processing in the estimation server 104. Specifically, in a case where an estimation program including the estimation model is executed, the estimation is performed by the CPU 220 and the GPU 223 collaborating to perform calculation. Alternatively, the calculation for the estimation processing may be performed only by the CPU 220 or the GPU 223. The GPU 223 is also used for learning processing.

A NIC 224 is used to communicate with the client terminal 102 and the data server 105 via the local network 106. An input unit 225 is composed of a keyboard, a mouse, and the like for operating the estimation server 104.

A display unit 226 displays input statuses and the like of the estimation server 104.

A system bus 227 connects the constituents 220 to 226 of the estimation server 104 described above.

Next, the configuration of data server 105 will be described.

A CPU 228 controls the entire data server 105. A HDD 229 stores programs and image data used by the CPU 228 for operating the data server 105. A RAM 230 is used to temporarily expand the program read from the HDD 229 by the CPU 228, execute the program expanded thereon, and temporarily hold the operational data.

A NIC 231 is used to communicate with the client terminal 102 and the estimation server 104 via the local network 106. An input unit 232 is composed of a keyboard, a mouse, and the like for operating the data server 105.

A display unit 233 displays input statuses and the like of the data server 105.

A system bus 234 connects the constituents 228 to 233 of the data server 105 described above.

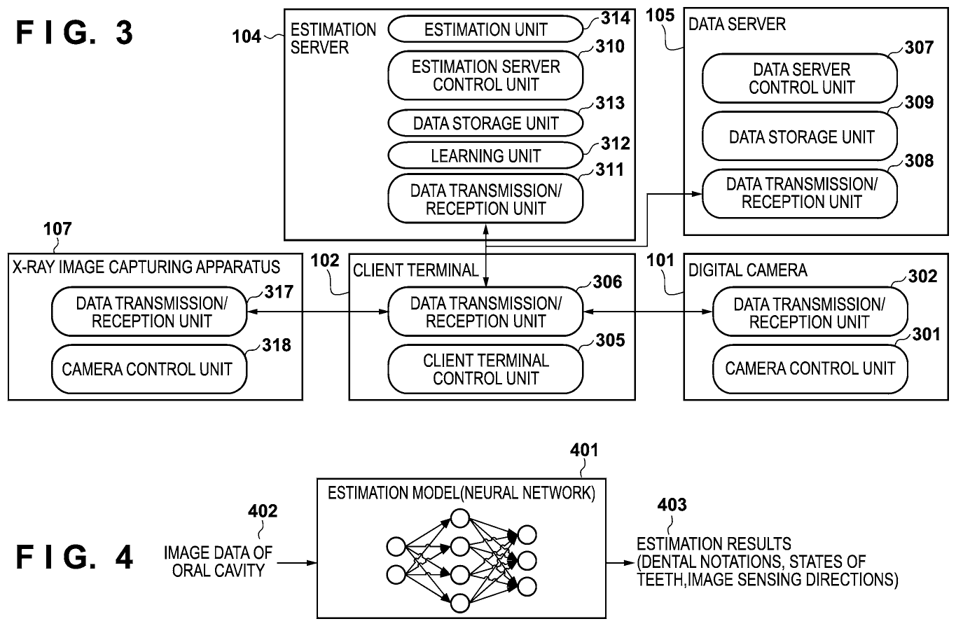

Next, with reference to FIG. 3, the functional configuration of each apparatus realized by using the hardware shown in FIG. 2 and a program will be described.

The CPU 201 reads a program for controlling the digital camera 101 from the ROM 202, and expands a part of the program to the RAM 203, thereby a camera control unit 301 of the digital camera 101 controls the entire digital camera 101. For example, the camera control unit 301 performs controls such as to cause the camera engine 209 to process an image input from the image sensor and cause the display unit 208 to display an image stored in the storage unit 210 according to the user's operation from the data server 105 and the input unit 207.

A data transmission/reception unit 302 transmits/receives data to/from the client terminal 102 via the I/F unit 206.

The CPU 235 reads a program for controlling the X-ray image capturing apparatus 107 from the ROM 236, and expands a part of the program to the RAM 237, thereby a camera control unit 318 of the X-ray image capturing apparatus 107 controls the entire X-ray image capturing apparatus 107. For example, the camera control unit 318 performs controls such as to cause the camera engine 241 to process an image input from the image sensor and cause the display unit 240 to display an image stored in the storage unit 242 according to the user's operation from the data server 105 and the input unit 239.

A data transmission/reception unit 317 transmits/receives data to/from the client terminal 102 via the I/F unit 243.

The CPU 212 reads a program for controlling the client terminal 102 from the HDD 213, and expands a part of the program to the RAM 214, thereby a client terminal control unit 305 of the client terminal 102 controls the entire client terminal 102.

A data transmission/reception unit 306 receives image data transmitted from the digital camera 101 and the X-ray image capturing apparatus 107 via the I/F unit 216, and transmits the image data to the estimation server 104 and the data server 105 via the NIC 215.

The CPU 220 reads a program for controlling the estimation server 104 from the HDD 221, and expands a part of the program to the RAM 222, thereby an estimation server control unit 310 of the estimation server 104 controls the entire estimation server 104.

A data transmission/reception unit 311 transmits/receives image data, learning data, etc. to/from the client terminal 102 and the estimation server 104 via the NIC 224.

A learning unit 312 performs learning processing using the GPU 223 and/or the CPU 220 using the data held in the RAM 222 or the HDD 221. Here, images of an oral cavity including the teeth captured in advance, and information indicating the dental notations and the conditions (states of dental caries, etc.) of the teeth in the images, and the directions of image sensing (from which direction each image is sensed) are stored as a set in RAM 222 or HDD 221 as learning data. Then, the learning unit 312 learns using the images of the oral cavity including the teeth as input data, and information indicating the dental notations and the conditions (the states of dental caries, etc.) of the teeth in the images, and the directions of image sensing (from which direction each image is sensed), which is associated with the images as training data. Here, the images of the oral cavity including the teeth include visible light images and X-ray images. A data storage unit 313 stores the estimation model generated by the learning in the learning unit 312 in the HDD 221. When an image of the oral cavity including teeth is input, an estimation unit 314 uses the estimation model stored in the HDD 221 to estimate the dental notations and the conditions (states of dental caries, etc.) of the teeth in the image, and the directions of image sensing (from which direction the image was sensed). Here, as described above, the image of the oral cavity including the teeth includes a visible light image and an X-ray image. That is, by using the estimation model described in the present embodiment, regardless of whichever of a visible light image or an X-ray image is input, the dental notations and the conditions (states of dental caries, etc.) of the teeth in the image are estimated.

The CPU 228 reads a program for controlling the data server 105 from the HDD 229, and expands a part of the program to the RAM 230, thereby a data server control unit 307 of the data server 105 controls the entire data server 105.

A data transmission/reception unit 308 transmits/receives image data, learning data, etc. to/from the client terminal 102 and the estimation server 104 via the NIC 224. A data storage unit 309 stores the learning data in the HDD 229.

Next, with reference to FIG. 4, the contents estimated by the estimation model in the learning unit 312 will be described.

An estimation model 401 is an estimation model using a neural network or the like, and image data 402 is image data of images captured by the digital camera 101 or the X-ray image capturing apparatus 107 and input to the estimation model 401. Estimation results 403 are estimation results in a case where the image data 402 is input to the estimation model 401, and the dental notations and the conditions (states of dental caries, etc.) of the teeth in the image, and the direction of image sensing (from which direction each image was sensed) are estimated.

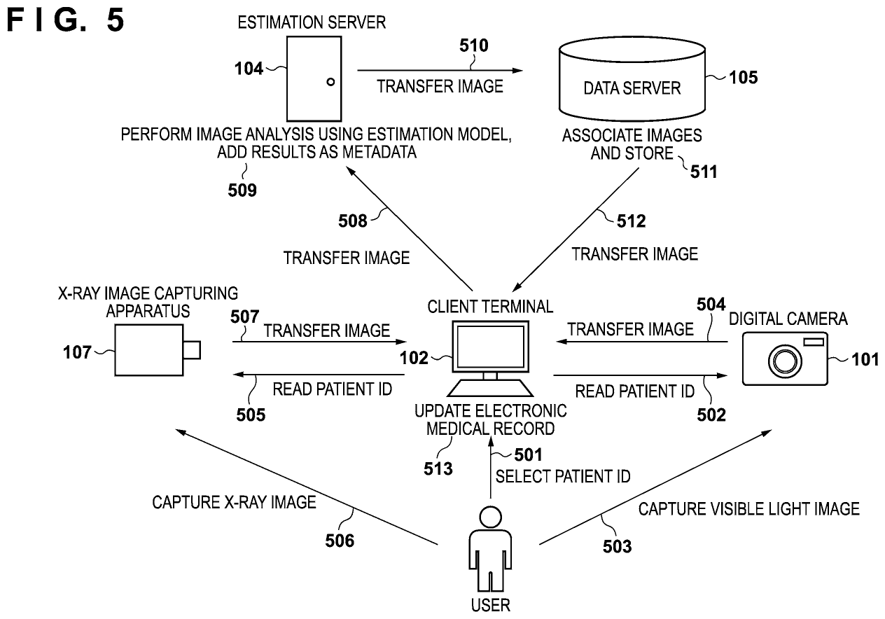

Next, with reference to FIG. 5, the flow of data in the system of the present embodiment using the estimation model shown in FIG. 4 will be described.

First, the user selects the patient ID at the client terminal 102 (501). When capturing a visible light image with the digital camera 101, the digital camera 101 reads the patient ID from the client terminal 102 (502). Then, based on the user's image sensing instruction, the digital camera 101 captures a visible light image (503) and transfers the captured visible light image to the client terminal 102 (504).

For capturing an X-ray image using the X-ray image capturing apparatus 107, the X-ray image capturing apparatus 107 reads the patient ID from the client terminal 102 (505). Then, based on the user's image sensing instruction, the X-ray image capturing apparatus 107 captures an X-ray image (506) and transfers the captured X-ray image to the client terminal 102 (507).

The client terminal 102 transfers the visible light image and/or the X-ray image to the estimation server 104 (508). The estimation server 104 performs image analysis using the estimation model 401, adds obtained estimation results 403 as metadata to the visible light image and/or the X-ray image (509), and transfer the visible light image and/or the X-ray image to which the metadata is added to the data server 105 (510).

The data server 105 associates related images with each other based on the metadata information and saves them in the HDD 229 (511). For example, images having a common patient ID and image sensing direction are linked to each other. Then, the data server 105 transfers the visible light image and/or the X-ray image to which the metadata is added to the client terminal 102 (512).

The client terminal 102 updates the information of the electronic medical record held in the HDD 213 by using the received visible light image and/or the X-ray image (513).

### First Embodiment

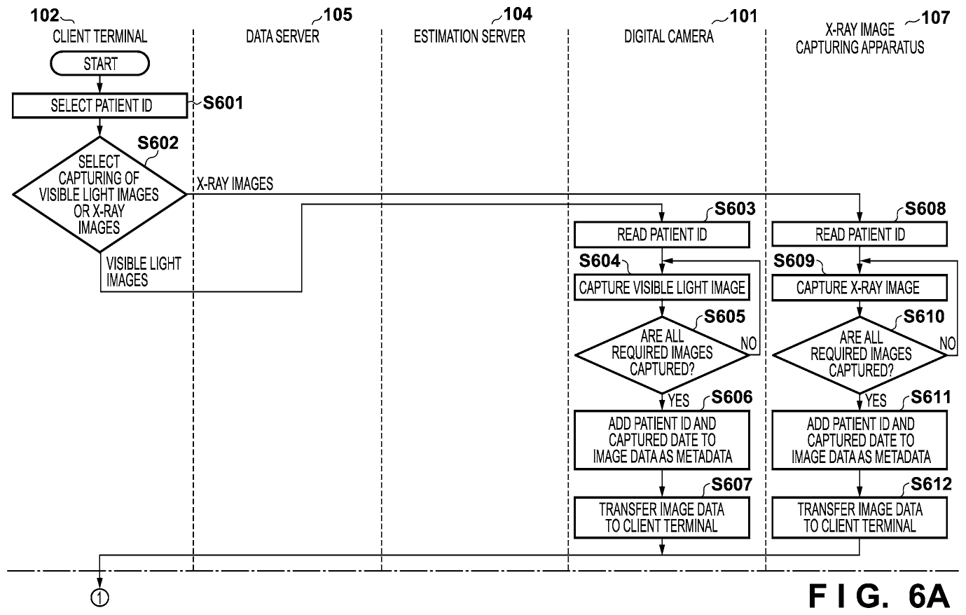

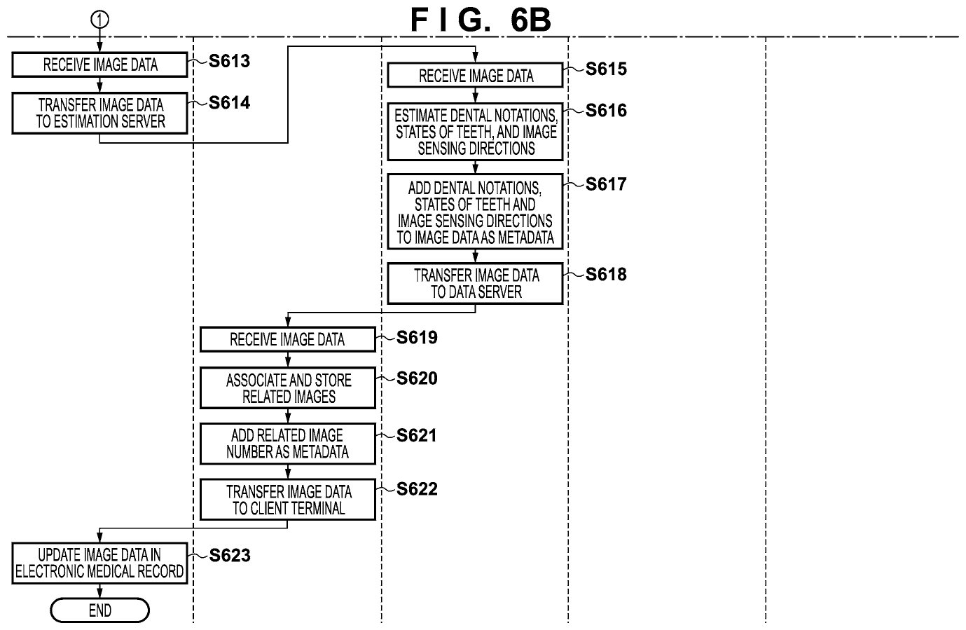

Hereinafter, with reference to FIGS. 6 to 8, the processing according to the first embodiment of the present invention performed in the information processing system having the above configuration will be described.

FIGS. 6A and 6B illustrate a flowchart showing the flow of the information processing according to the first embodiment. The processes in this flowchart are executed by the client terminal 102, data server 105, estimation server 104, digital camera 101, and X-ray image capturing apparatus 107.

In step S601, the client terminal 102 selects the patient ID based on the user input to the input unit 217. In step S602, the client terminal 102 prompts the user to choose whether to capture visible light images or X-ray images. If capturing of visible light images is selected, the process proceeds to step S603, and if capturing of X-ray images is selected, the process proceeds to step S608.

In step S603, the digital camera 101 reads the patient ID selected in step S601, and in step S604, executes capturing of a visible light image based on a user's image sensing instruction. In step S605, the digital camera 101 determines whether all of the required visible light images are captured, and if yes, the process proceeds to step S606, and if no, the process returns to step S604 and continues to capture a next visible light image.

In step S606, the digital camera 101 writes the patient ID and capturing date in the metadata area of the image data of the captured visible light images, and in step S607, transfers the image data of the visible light images to the client terminal 102.

Since processes of steps S608 to S612 are the same as the processes of step S603 to S607 performed in the digital camera 101, respectively, except that the X-ray image capturing apparatus 107 captures X-ray images, the description thereof will be omitted.

In step S613, the client terminal 102 receives the image data from the digital camera 101 or the X-ray image capturing apparatus 107, and in step S614, transfers the image data to the estimation server 104.

In step S615, the estimation server 104 receives the image data, and in step S616, performs image analysis based on the estimation model 401 to estimate dental notations and the conditions (states of caries, etc.) of the teeth in the images, and the image sensing directions (from which direction the images were sensed) of the images. In step S617, the estimation server 104 writes the estimation results 403 to the metadata area of the image data, and in step S618, transfers the image data to which the metadata is added to the data server 105.

In step S619, the data server 105 receives the image data, and in step S620, associates the related images and stores them in the HDD 229. For example, images having a common patient ID and image sensing direction are associated to each other. In step S621, the data server 105 writes the related image number in the metadata area of the image data of each image, and in step S622, transfers the image data to the client terminal 102.

In step S623, the client terminal 102 receives the image data and updates the image data in the electronic medical record.

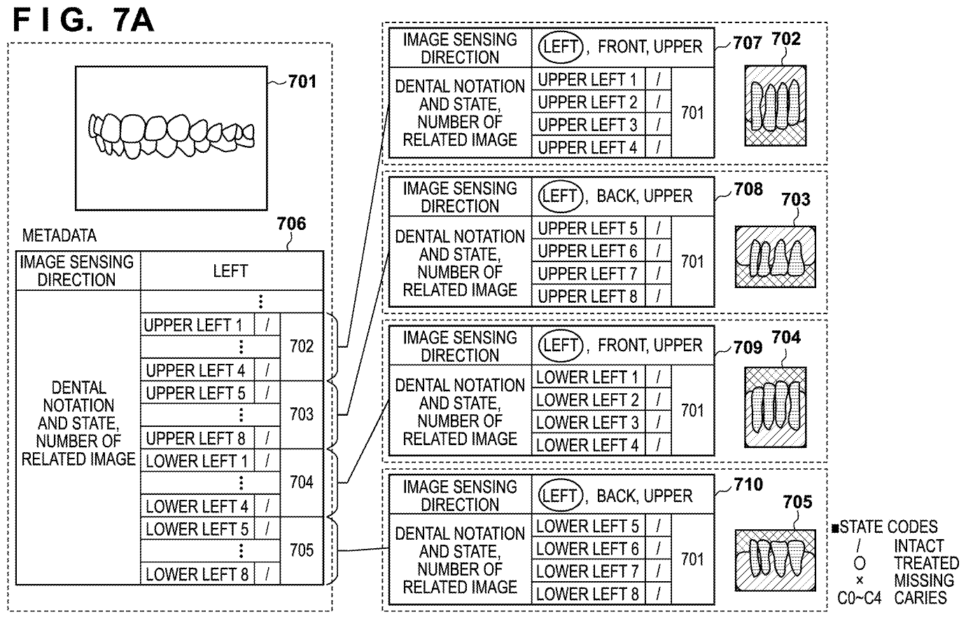

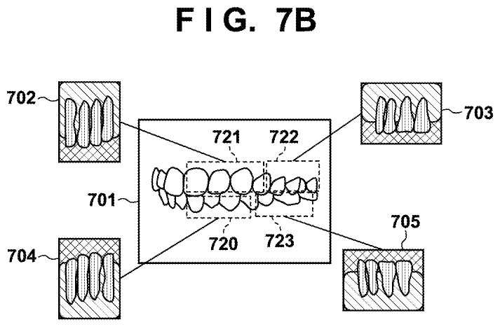

FIGS. 7A and 7B are diagrams for explaining how to associate images according to the first embodiment.

In FIG. 7A, the reference numeral 701 represents a visible light image of an oral cavity captured by the digital camera 101; 702 to 705, X-ray images of the oral cavity captured by the X-ray image capturing apparatus 107; and 706 to 710, a part of the information written in the metadata areas of the visible light image 701 and the X-ray images 702 to 705, respectively.

In each metadata area, information on the dental notations and the conditions (states of caries, etc.) of the teeth in each image, and the image sensing direction (from which direction the image is sensed) are written, and the images having matching information are associated to each other. For example, since the metadata areas 706 and 707 of the visible light image 701 and the X-ray image 702, respectively, include common dental notations “upper left 1 to upper left 4”, these images are associated with each other. The data server 105 assigns an associated image number to the metadata area of each image.

In the present embodiment, an example of associating images by matching the dental notations is given, however, images having the information of the same image sensing direction may be associated with each other.

In FIG. 7B, reference numerals 720 to 723 are diagrams showing examples of frames superimposed on the visible light image 701, and indicate regions corresponding to the X-ray images 702 to 705, respectively. Display/non-display of the frames 720 to 723 may be arbitrarily selected by the user.

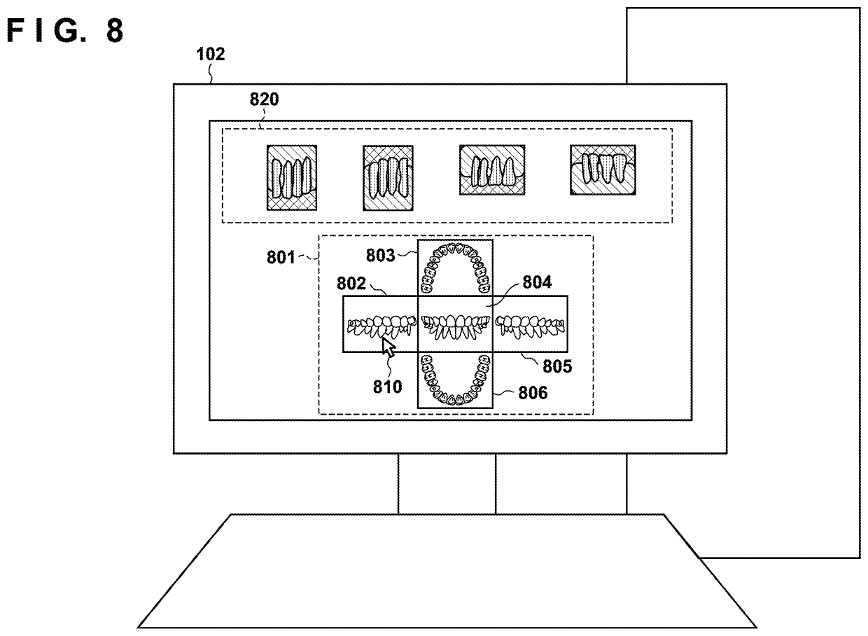

FIG. 8 is a diagram showing an example of a user interface displayed on the client terminal 102 according to the first embodiment.

In FIG. 8, a reference numeral 801 denotes a visible light image display area for showing visible light images of the oral cavity captured by the digital camera 101. In the visible light image display area 801, a visible light image 802 is of the patient's teeth sensed from the right side, a visible light image 803 is of the patient's upper teeth, a visible light image 804 is of the patient's teeth sensed from the front, a visible light image 805 is of the patient's teeth sensed from the left side, and a visible light image 806 is of the patient's lower teeth. A reference numeral 810 denotes a cursor that can be operated by the input unit 217. FIG. 8 shows a state in which the user has selected the visible light image 802.

A reference numeral 820 denotes an X-ray image display area for showing X-ray images of the oral cavity captured by the X-ray image capturing apparatus 107. In the X-ray image display area 820, the X-ray images associated with the visible light image selected by the user using the cursor 810 are displayed. In the present embodiment, since the user has selected the visible light image 802, four X-ray images associated with the visible light image 802 are displayed in the X-ray image display area 820.

As described above, according to the first embodiment, the visible light images and the X-ray images can be associated and managed based on the metadata added to the images.

### Second Embodiment

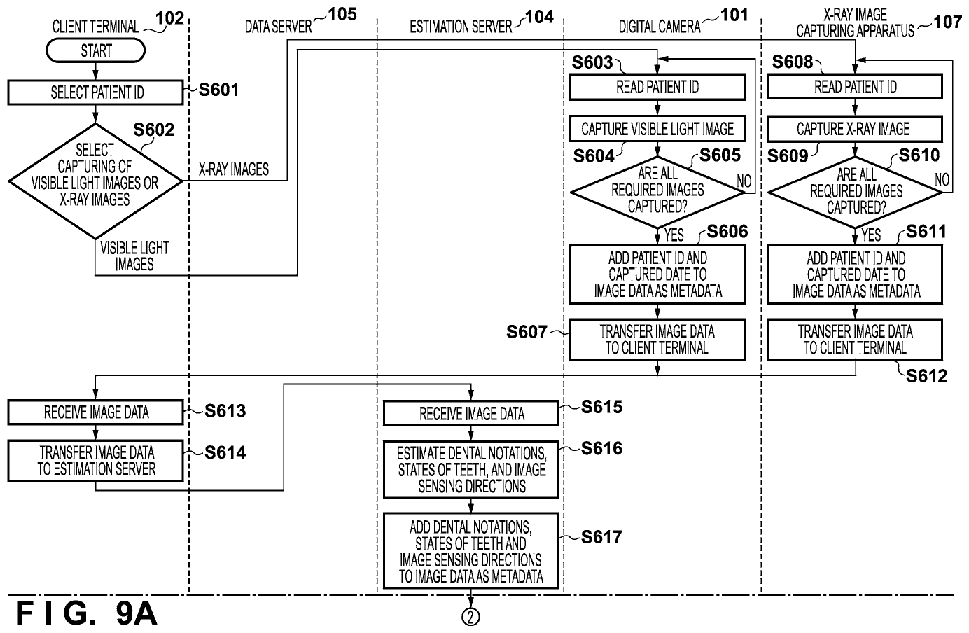

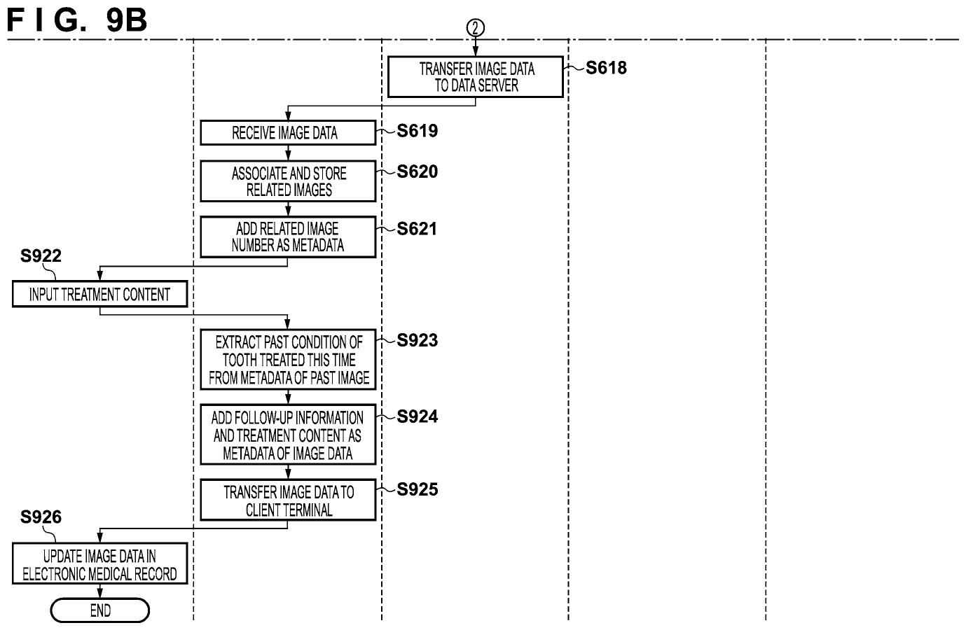

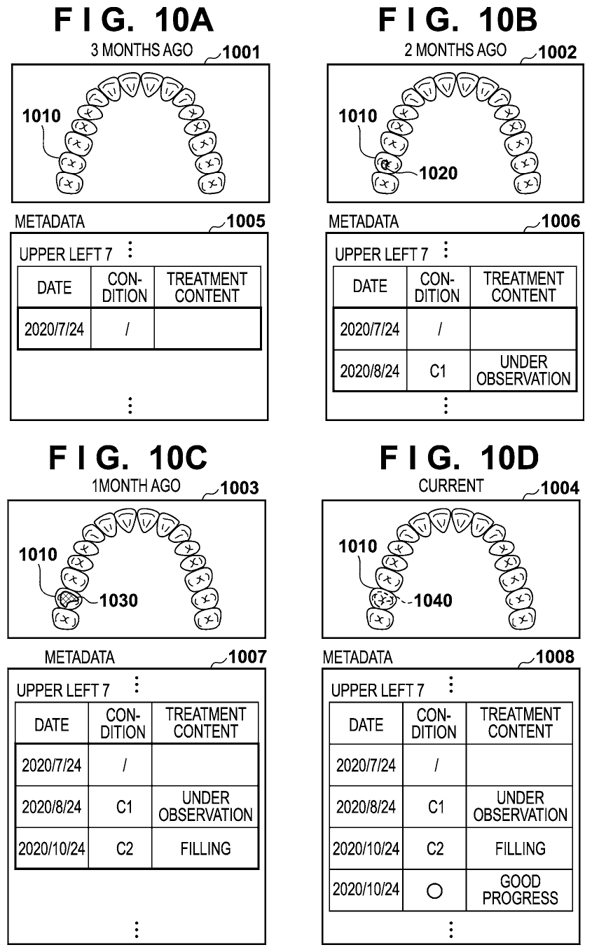

Next, with reference to FIGS. 9A and 9B and FIGS. 10A to 10D, the processing according to the second embodiment of the present invention performed in the information processing system described with reference to FIGS. 1 to 5 will be described.

FIGS. 9A and 9B illustrate a flowchart showing the flow of information processing according to the second embodiment. The processes in this flowchart are executed by the client terminal 102, data server 105, estimation server 104, digital camera 101, and X-ray image capturing apparatus 107.

In FIGS. 9A and 9B, the same step numbers are assigned to the same processes as the processes of step S601 to S621 described with reference to FIGS. 6A and 6B of the first embodiment, and the description thereof will be omitted.

In step S621, when the data server 105 assigns a related image number to the metadata area of the image data of each image, in step S922, the client terminal 102 accepts inputs of the treatment content performed by the user (dentist) to the patient. The user can input the treatment content using the input unit 217 while looking at the display unit 218.

In step S923, the data server 105 extracts the past condition of the tooth treated this time from the metadata of the past image/images. In step S924, the data server 105 writes the treatment content input in step S922 and the past condition extracted in step S923 into the metadata area of the image data of the latest image.

In step S925, the data server 105 transfers the image data to which the metadata is added to the client terminal 102. In step S926, the client terminal 102 takes in the image data and updates the image data of the electronic medical record.

FIGS. 10A to 10D are explanatory views of adding follow-up information according to the second embodiment.

FIGS. 10A to 10D are diagrams showing visible light images 1001 to 1004 in an oral cavity captured 3 months ago, 2 months ago, 1 month ago, and this time, respectively, and part of information 1005 to 1008 written in the metadata areas of visible light images 1001 to 1004. A reference numeral 1010 indicates a specific tooth. In the present embodiment, the tooth 1010 is defined as dental notation “upper left 7”, and the description will be given focusing on the follow-up information about “upper left 7”.

As shown in FIG. 10A, since there is no caries in the tooth 1010 at the stage when the visible light image 1001 is captured “/ (intact)” is recorded in the “condition” of the metadata area.

In the visible light image 1002 shown in FIG. 10B, a reference numeral 1020 represents a carious portion of the tooth 1010. At this time, the progress of the carious portion 1020 was “C1 (mild caries)”, and the result of the dentist's examination was “under observation”, so that the following information was written in the metadata area.
Information related to “upper left 7” written in the metadata 1005
The condition of “upper left 7” (C1) and treatment content (under observation) on the image sensing date of the visible light image 1002

In the visible light image 1003 shown in FIG. 10C, a reference numeral 1030 represents a carious portion of the tooth 1010. At this time, the progress of the carious portion 1030 was “C2 (mild, but treatment needed)”, and the treatment content performed by the dentist was “shave the carious portion and fill it”, so that the following information was written in the metadata area.
Information related to “upper left 7” written in the metadata 1006
The condition of “upper left 7” (C2) and treatment content (filling) on the image sensing date of the visible light image 1003

In the visible light image 1004 shown in FIG. 10D, a reference numeral 1040 represents a treatment scar on the tooth 1010. At this time, the state of the tooth 1010 is “o (treated)”, and the result of the dentist's examination is “good progress”, so that the following information is written in the metadata area.
Information related to “upper left 7” written in the metadata 1007
The condition of “upper left 7” (o) and the examination result (good progress) on the image sensing date of the visible light image 1004

By adding “past information” and “latest medical examination result” to an image in this way, it is possible to retroactively acquire follow-up information by looking at the metadata area of the latest image.

As described above, according to the second embodiment, it is possible to manage the visible light images and the X-ray images in association with each other based on the metadata given to the images, and obtain follow-up information from the metadata given to the latest image.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-094576, filed Jun. 4, 2021 which is hereby incorporated by reference herein in its entirety.

What is claimed is:

1. An information processing system comprising one or more processors and/or circuitry which functions as:
an information processor capable of transmitting and receiving data; and
an estimation unit that estimates dental notations of teeth in each of visible light images and X-ray images of oral cavities input through the information processor and estimates image shooting direction of each of the visible light images and the X-ray images,
wherein the information processor adds the dental notations and the image sensing direction to each of the visible light images and the X-ray images as metadata, and manages the visible light images and the X-ray images by associating the visible light images and the X-ray images using the metadata.

2. The information processing system according to claim 1, wherein the information processor associates the visible light image and the X-ray image having a common dental notation as the metadata.

3. The information processing system according to claim 1, wherein the information processor associates the visible light image and the X-ray image having a same image sensing direction as the metadata.

4. The information processing system according to claim 1 further comprising an input unit used for inputting information on a patient,
wherein the information processor adds the information on the patient to each of the visible light images and the X-ray images as the metadata, and manages the visible light image/images and the X-ray image/images for each patient.

5. The information processing system according to claim 4, wherein the information processor further adds date of capturing each of the visible light images and the X-ray images, and information on condition of teeth input by the input unit to each of the visible light images and the X-ray images as the metadata and manages the metadata.

6. The information processing system according to claim 1, further comprising a display unit that displays the visible light image/images, the X-ray image/images and the metadata which are managed in association with each other.

7. The information processing system according to claim 6, wherein a frame indicating an area corresponding to each X-ray image related to the visible light image/images displayed on the display unit is superimposed on the visible light image/images.

8. The information processing system according to claim 1, wherein the information processor and the estimation unit are formed on different devices.

9. The information processing system according to claim 1, wherein the information processor and the estimation unit are formed on the same device.

10. The information processing system according to claim 1 further comprising:
a first image sensing unit that senses a visible light image of an oral cavity; and
a second image sensing unit that senses an X-ray image of an oral cavity,
wherein the information processor obtains the visible light images from the first image sensing unit and the X-ray images from the second image sensing unit.

11. An information processing method comprising:
inputting visible light images and X-ray images of oral cavities; and
estimating dental notations of teeth in each of the visible light images and the X-ray images and estimating image shooting direction of each of the visible light images and the X-ray images,
the dental notations and the image sensing direction are added to each of the visible light images and the X-ray images as metadata, and the visible light images and the X-ray images are managed by associating the visible light images and the X-ray images using the metadata.

12. A non-transitory computer-readable storage medium, the storage medium storing a program that is executable by the computer, wherein the program includes program code for causing the computer to function as an image processing system comprising:
an information processor capable of transmitting and receiving data; and
an estimation unit that estimates dental notations of teeth in each of visible light images and X-ray images of oral cavities input through the information processor and estimates image shooting direction of each of the visible light images and the X-ray images,
wherein the information processor adds the dental notations and the image sensing direction to each of the visible light images and the X-ray images as metadata, and manages the visible light images and the X-ray images by associating the visible light images and the X-ray images using the metadata.
